(19) United States
(12) Patent Application Publication
(10) Pub. No.: US 2026/0295169 A1
(43) Pub. Date: Oct. 1, 2026

(54) FINGER GRIP

(71) Applicant: Terumo Europe NV, Leuven (BE)

(72) Inventors: Nicolas EON, Strasbourg (FR); Jésus ZAMORANO, Oyonnax (FR); Dominique MALFROY, Longchaumois (FR); Pierre ROY, Paris (FR)

(21) Appl. No.: 18/881,375

(22) PCT Filed: Jul. 7, 2023
(86) PCT No.: PCT/EP2023/068909

§ 371 Date: Jan. 6, 2025

Related U.S. Application Data
(60) Provisional application No. 63/358,987, filed on Jul. 7, 2022.

(51) Int. Cl. A61M 5/31; A61L 2/20; A61L 103/15

(52) U.S. Cl. CPC A61M 5/3137; A61L 2/20; A61L 2103/15; A61M 2005/3139; A61M 2202/0007; A61M 2205/586; A61M 2207/00; A61M 2209/06

(57) ABSTRACT

There is provided an injection device (1) comprising a syringe (10) having a barrel (11) and a flange (12); and a finger grip (100) coupled with the syringe. The finger grip comprises a base (104) receiving the flange and a collar (103) receiving an end portion of the barrel adjacent to the flange. An inner surface of the base part (110,120) and the collar (130) respectively provide a first set of first point and/or line contacts (C1) for engaging with an exterior surface of the flange and a second set of second point and/or line contacts (C2) for engaging with an exterior surface of the barrel. The inner surface is smooth or free of outwardly extending protrusions for contacting the syringe. The contact may be loose with respect the flange and/or the barrel.

### TECHNICAL FIELD AND BACKGROUND

Aspects of the present disclosure relate to an injection device for drug delivery. In particular, the disclosure generally relates to a finger grip accessory for associating with the injection device, to an assembly of the injection device and the finger grip accessory associated thereto, a packaged, sterilized injection device pre-filled with a medicament, and methods of manufacturing.

Syringes are medical delivery devices that are used to administer a medicament to a patient.

Syringes are often marketed either in prefilled form, wherein a set dosage of medicament is already provided therein, or they are empty and intended to be filled from a vial or other source of medicament by an end user at the time administration of the medicament is desired.

Syringes generally include a barrel portion for holding the medicament. The distal end of the barrel is often configured, typically by a hub, for connecting the barrel to a cannula. When the hub is connected to the syringe, a fluid connection between the needle and the syringe is established such that fluid from the syringe can be injected via the needle.

A plunger rod may be inserted through the open proximal end of the syringe barrel. A user can administer the medicament by applying a manual force to the plunger.

Commercial syringes are often provided with flange. The flange is generally provided around the open proximal end of the syringe barrel and extends radially therefrom to provide a form of finger rest for manipulation of the device.

To improve grip, usability, and/or ergonomics of the syringe, it is known to couple a finger grip component to the device. Finger grips are sometime also referred to as a “backstop”. The finger grip may have a radial length that is larger than a length of the flange of the barrel. Alternatively, or in addition, the finger grip may provide a more ergonomic surface to the user, e.g. for reducing user-fatigue. To provide a stable connection with the device the finger grip is generally configured to engage with one or more of the flange or a portion of the barrel.

Finger grips may be provided in a number of configurations. Known variations include components configured to couple with the device along an axial direction of the barrel, e.g. by sliding along the barrel from its proximal end towards the flange. Components that couple to the device from a radial direction are also known, e.g. by snapping onto the barrel and/or by sliding along the radially extending flange. Alternative variations include multi-component grips that can be assembled (e.g. clicked) around a syringe.

For integrity of the medicament and for patient safety it is important to sufficiently sterilize the syringe. Sterilization may occur at several stages. For example, in the assembly process, including pre-fill stages (e.g., sterilization of the empty barrel and/or plunger) and post-fill stages (e.g., external sterilization of the assembled pre-filled syringe).

External sterilization typically occurs after the syringe has been fully assembled and optionally pre-filled, and/or at least partially positioned in its final packaging.

External sterilization can pose a number of challenges/limitations. For example, sterilization by radiation or temperature may not be appropriate in some case, e.g. for sensitive medicaments. For sterilization by fluid disinfectants (typically gasses such as steam, NOx, or ethylene oxide) it may be difficult to achieve a desired or required level of sterilization for external surfaces of the syringe and/or assembled components. In particular it remains a challenge to effectively sterilize surfaces between the syringe and parts, such as a finger grip, coupled thereto.

US 20210060258A1 discloses an injection device including a syringe having a barrel and a flange and a backstop configured to be coupled with the syringe adjacent to the flange. The backstop includes an inner surface generally extending around at least a portion of the syringe, wherein the inner surface includes at least one protrusion extending away from the inner surface and configured to engage the flange and/or the barrel to permit or promote airflow through a space between the inner surface and the syringe. The protrusions can contribute to local reduction of occluded surfaces areas between the backstop and the barrel and/or the flange.

### SUMMARY

The present disclosure aims to provide a solution having further improved gas access between the syringe and finger grip accessory when associated therewith, while providing at least acceptable or even improved mechanical stability during normal use. As detailed herein below the disclosure provides an injection device having reduced or even eliminated occluded exterior surface areas. The device further offers: compatibility with conventional mechanized assembly lines and/or relaxed (mass)manufacturability of the grip part, particularly with molding methods.

Aspects of the present disclosure relate to a finger grip accessory for associating with the injection device and to an assembly of the syringe and the finger grip accessory. Further, the disclosure provides a packaged injection product, specifically an assembly of the finger grip accessory and the syringe device. The syringe can be pre-filled with a medicament and/or exposed to a gaseous sterilizing agent even when prefilled or partly packaged. The disclosure further provides a method of manufacturing the grip and a method of manufacturing the packaged injection product.

Accordingly, according to a first aspect there is provided an injection device comprising a syringe having a barrel and a flange; and a finger grip coupled with the syringe. The syringe may be pre-filled with a medicament. Alternatively, the syringe can be empty.

The finger grip comprises at least a base part defining a first portion of a cavity of the grip receiving the flange, and a collar part defining a second potion of the cavity of the grip, receiving an end portion of the barrel adjacent the flange. To improve an ergonomics, tactile, and or handleability of the device the grip can be provided with a first and second finger rest portions that extend laterally from the grip, e.g. from a side of the base and/or the collar.

To provide a mechanically reliable coupling between the finger grip and the syringe, an inner surface of the base part bounding the first portion of the cavity and an inner surface of the collar bounding the second portion of the cavity are formed such as to respectively provide a first set of first contacts for engaging with an exterior surface of the flange, and a second set of second contacts for engaging with an exterior surface of the barrel. It will be appreciated that contacts comprised the first and second set are distributed around a berth for receiving the barrel, respectively for the flange. For example, so as to provide a stable coupling with the flange the first set of contacts includes contacts arranged for engaging bottom and top faces of the flange engaged. The contacts typically distributed to preclude flush contact areas during full coupling. To reduce accidental release of the grip, e.g. during operation, the barrel berth can be configured to provide a snap fit relation.

To minimize an overall contact area between grip and syringe the contacts can be configured as what is referred to as a line contact, a point contact, or a combination thereof. Configuring the grip such that a coupling with the syringe is realized by point and/or line contacts advantageously minimizes an overall contact area, specifically flush contact area, between grip and syringe. Accordingly, even in a coupled state exterior surfaces of the syringe and grip as the finger grip can advantageously remain available for gas access by passages/spaces formed between the contacts, e.g. for sterilization with a gaseous agent such as NOx, ethylene oxide or any other suitable agent known in the field. In some variations all of the first and the second contacts are comprised of line contacts. The orientation of the line contacts is preferably such that: at the collar the contacts are aligned (parallel) with an axial direction of the barrel, and that at the base, the lines are aligned with a sideways coupling direction/sliding motion of the grip onto the syringe.

In strongly preferred variations the inner surface surrounding the cavity, specifically at least respective inner surfaces of the base part of the grip and the inner surface of the base part of the grip are smooth or at least free of outwardly extending protrusions for contacting portions of the syringe, over zones that least encompass the contacts comprised in the first and the second set. Reducing a number of outwardly extending protrusions and/or providing a smooth surface finish advantageously reduces a number of nooks, ridges, irregulates, and/or shielded surface portions for contaminants to adhere to, e.g. at edges or abrupt angles at a base of an outwardly extending protrusion. Additionally, minimizing roughness and/or or eliminating presence of protrusions relaxes one or more stages of manufacturing the grip, including filling and release from a mould.

Advantageously, the grip can be manufactured as a single integrated part as opposed an assembly of two or more assembled (e.g. glued) sub parts. It will be understood that the term smooth includes surface having a gradually sloping surface, a bend, a fold and/or intersects of two or more sloped surfaces (e.g. intersects of adjacent planar facets).

To increase handleability the grip can comprise one or more finger rest portion. As known in the field these portions increase accessible surface area for manual handling of the device. To this end the grip can provide at least a first and second finger rest portion. The finger rest portions can for example extend laterally from the grip, e.g. from the collar or the base portion.

Advantageously, engagement between the grip and the syringe can be provided exclusively by the contacts, or by a subset thereof, as comprised in first and second set of contacts. In this way it can be realized that, during normal use, opposing faces of the grip and syringe (excluding the line or point contacts) are separated by a gap.

In some variations the grip can be configured, shaped, dimensioned, such that a majority or even of all of the contacts actually engage the syringe. However, it will be appreciated that not all contacts need to partake in simultaneous engaging the syringe when coupled.

In some variations there remains a separation distance between some of the contacts and the syringe when fully coupled. For example, between the flange and an apex of a contact at a portion of the base part facing a top face of said flange.

Specifically, in one variation there is a tight coupling between the flange of the syringe and the base part of the grip and a looser coupling between the collar of the grip and the barrel of syringe. In another variation the grip is configured such that there is a tight coupling between the barrel of the syringe and the collar part of the grip and a looser coupling between the flange of the syringe and the base part of the grip. Inventors found that for both configurations mechanical stability can be excellent and that slack between corresponding parts can be essentially unnoticeable or at least acceptable during normal operation.

Accordingly, in a preferred variation there is a tight coupling between the flange of syringe and the grip by the base part being configured to provide a simultaneous direct contact of each the first contacts comprised in the first subset with the exterior surface of the flange, and wherein there is a looser coupling between the barrel of syringe and the grip by the collar part being configured such that only limited subsets of the second contacts comprised in the second set can be in a simultaneous direct contact with the barrel during normal operation.

In another preferred variation, wherein there is a tight coupling between the barrel of syringe and the grip by the collar part being configured to provide a simultaneous direct contact of each the second contacts comprised in the second subset with the exterior surface of the barrel, and wherein there is a looser coupling between the flange of syringe and the grip by the base part being configured such that only limited subsets of the first contacts comprised in the first set can be in a simultaneous direct contact with the flange during normal operation.

A maximum slack (distance) between non-engaging first contacts and the flange or the non-engaging second contacts and the barrel affects gas flow resistance between the parts, e.g. during sterilization. The larger the slack the lower the flow resistance can be. To minimize potentially affecting a mechanical stability of the coupling, the slack is preferably no more than about 0.8 mm. Preferably, below 0.4 mm, most preferably in a range of 50-300 μm.

In yet another variation there is a loose coupling to both the flange and the barrel. In such variations the grip is configured such that only limited subsets of the first and second contacts as comprised in the first and second set can be in a simultaneous direct contact with the flange, respectively the barrel during normal operation. Inventors find that configurations where the coupling to both the barrel and flange is some loose (not tight) can further increase gas access between syringe and flange while still providing acceptable mechanical stability, especially so long as slack does not exceed 0.8 mm, preferably≤0.4 mm, most preferably≤0.3 mm.

An overall number of first contacts for engaging the flange is preferably as low as possible. Advantageously, the number can be as low as four. Typically, the set of first contacts comprise two (or more) contacts for engaging a top face of the flange and two (or more) contacts for engaging a bottom face of the flange.

An overall number of second contacts for engaging the barrel is preferably as low as possible. Generally, a number of line contacts comprised in the second set is ≤10. Preferably, ≤7. Advantageously the number of second contacts for engaging the barrel can as low as 5 or even 3 without compromising on mechanical stability of the coupling.

In some variations the grip further comprises a backstop, e.g. a tab, to restrict release of a plunger from the barrel.

The disclosure further provides a finger grip configured for coupling with a syringe comprising a barrel and a flange, preferably the finger grip as defined herein. Said finger grip comprising at least: a base part of the grip defining a first portion of a cavity of the grip for receiving the flange, a collar part of the grip defining a second potion of the cavity of the grip, for receiving an end portion of the barrel adjacent the flange, and preferably also at least a first and second finger rest extending laterally from the grip, wherein an inner surface of the base part bounding the first portion of the cavity and an inner surface of the collar bounding the second portion of the cavity are formed such as to respectively provide a first set of first contacts for engaging with an exterior surface of the flange, and a second set of second contacts for engaging with an exterior surface of the barrel, and wherein the inner surface of the base part of the grip and the inner surface of the base part of the grip are smooth or at least free of outwardly extending protrusions for contacting portions of the syringe, over zones that least encompass the contacts comprised in the first and the second set of contacts.

### BRIEF DESCRIPTION OF DRAWINGS

These and other features, aspects, and advantages of the apparatus, systems and methods of the present disclosure will become better understood from the following description, appended claims, and accompanying drawing wherein:

FIG. 1 provides a side view of an injection device for drug delivery;

FIG. 2 provides a cross section view of an injection device;

FIG. 3 provides a top view of a finger grip;

FIG. 4 provides a cross section top view of an injection;

FIG. 5 provides a partial cross section side view of an injection device;

FIG. 6 provides a side view of a finger grip;

FIG. 7 provides a perspective top view of a finger grip;

FIG. 8 provides provide a perspective bottom view of a finger;

FIG. 9 provides a partial side view of an injection device for drug delivery;

FIG. 10 provides a partial cross section side view of an injection device;

FIGS. 11A and 11B provide cross section bottom views of an injection device;

FIGS. 12A and 12B provide a cross section bottom view of an injection device;

FIG. 13 provides a partial side view of an injection device;

FIGS. 14 and 15 provide detail views of the device shown in FIG. 13;

FIG. 16 provide a cross section bottom view of an injection device;

FIG. 17 provides a bottom view of a finger grip;

FIG. 18 provides a partial bottom view of a finger grip;

FIG. 19 provides a partial side of an injection device;

FIG. 20 provides a partial side of a finger grip; and

FIGS. 21A, 21B, and 21C provide side views of an injection device.

### DESCRIPTION OF EMBODIMENTS

Terminology used for describing particular embodiments is not intended to be limiting of the invention. As used herein, the singular forms “a”, “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features. It will be further understood that when a particular step of a method is referred to as subsequent to another step, it can directly follow said other step or one or more intermediate steps may be carried out before carrying out the particular step, unless specified otherwise. Likewise it will be understood that when a connection between structures or components is described, this connection may be established directly or through intermediate structures or components unless specified otherwise.

The invention is described more fully hereinafter with reference to the accompanying drawings, in which embodiments of the invention are shown. In the drawings, the absolute and relative sizes of systems, components, layers, and regions may be exaggerated for clarity. Embodiments may be described with reference to schematic and/or cross-section illustrations of possibly idealized embodiments and intermediate structures of the invention. In the description and drawings, like numbers refer to like elements throughout. Relative terms as well as derivatives thereof should be construed to refer to the orientation as then described or as shown in the drawing under discussion. These relative terms are for convenience of description and do not require that the system be constructed or operated in a particular orientation unless stated otherwise.

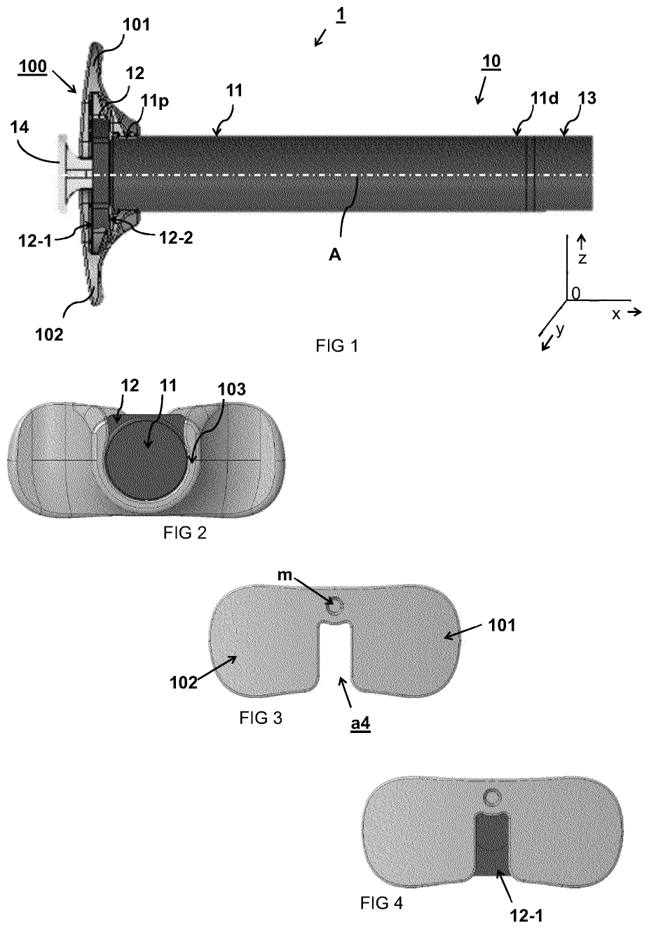

FIG. 1 provides a side view of an injection device 1 for drug delivery in accordance with the present disclosure. The device comprises a syringe 10, having a barrel 11 and a flange 12. The flange 12 is provided along a proximal end 11p of the barrel. The opposing distal end 11d is shown comprising a hub 13 for receiving a cannula (not shown). The device 1 further comprises a finger grip 100. The grip is depicted in a fully engaged position. The grip engages the flange from an upper 12-1 and a lower face 12-2 thereof. The grip also engages a portion of the exterior face of the barrel at the proximal end. The finger grip 100 is fitted around a plunger 14 inserted into the barrel. The grip does not contact the plunger during normal operation. The plunger can slide within the barrel along its central longitudinal axis A. Advantageously, and as best seen in FIG. 17, the grip can function as backstop, e.g. by tab 50. The backstop allows passage of plunger rod 14r but restricts motion of the plunger upon engaging with plunger base 14b.

Radially extending wing portions 101,102 of the finger grip part provide extended surface area for a user to handle the device. As detailed herein below the grip can be provided in a number of variations including variations with a tight connection to both barrel and flange, variations with a tight coupling with respect to the flange and a looser coping with the barrel, variations with a tight coupling with respect to the barrel and a looser coping with the flange, as wells as variations wherein the coupling to flange and barrel are both somewhat loose.

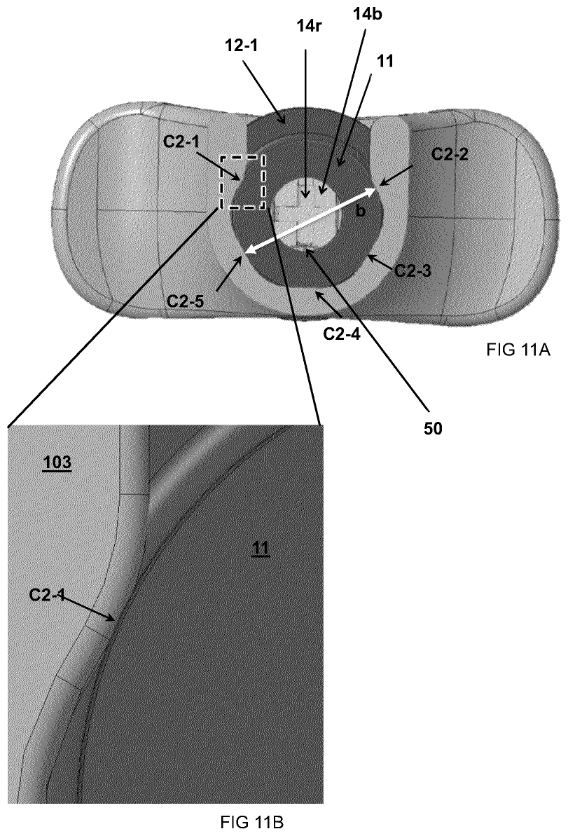

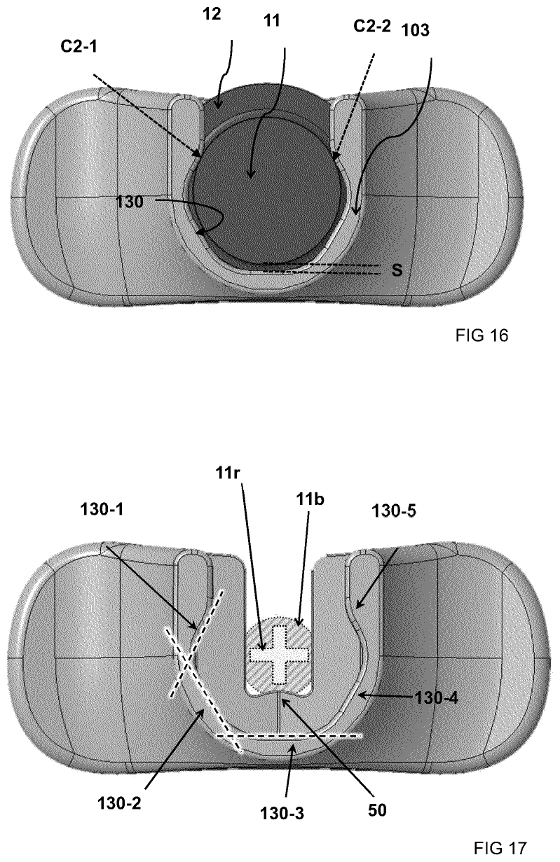

FIGS. 2-21 provide detailed views of aspects relating to embodiments of the injection device 1, the finger grip 10 and the coupling between grip and syringe, wherein FIGS. 11-15 detail aspects of embodiments configured to realize a tight coupling between the grip on one side and the barrel and flange on the other, and wherein FIGS. 16-21 illustrate aspects of variations having one looser coupling (flange or barrel) with the syringe. It will be appreciated that variations wherein the coupling to both barrel and flange are loose include the corresponding aspects as appropriate.

As best seen in e.g. FIGS. 5, 8 and 10, the grip 100 can, at least conceptually, be understood as comprising two interconnected parts including: a base part 104 providing a berth for receiving the flange 12 and a collar part 103 providing a berth for receiving the barrel 11. The berths are fluidly connected forming a continuous cavity.

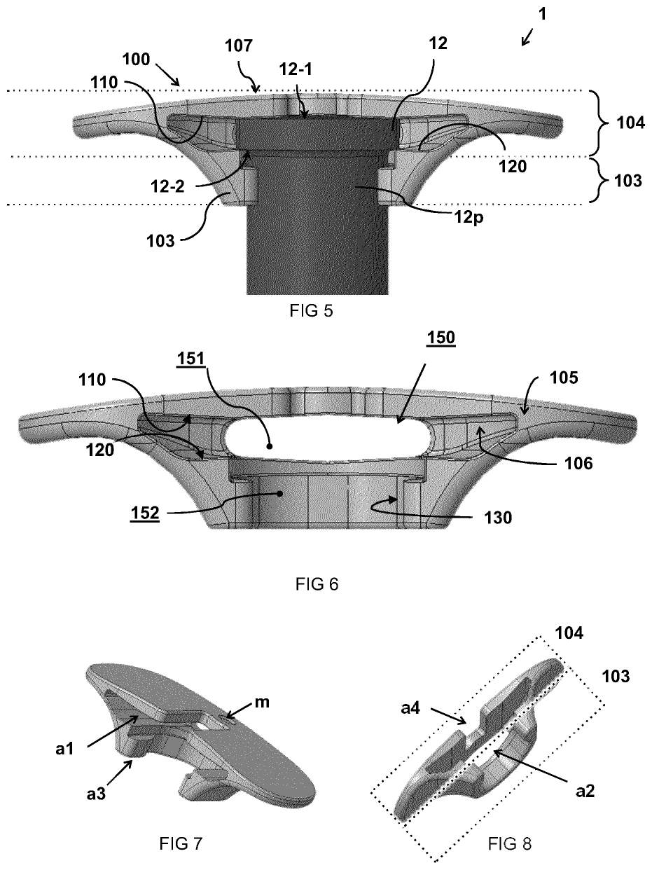

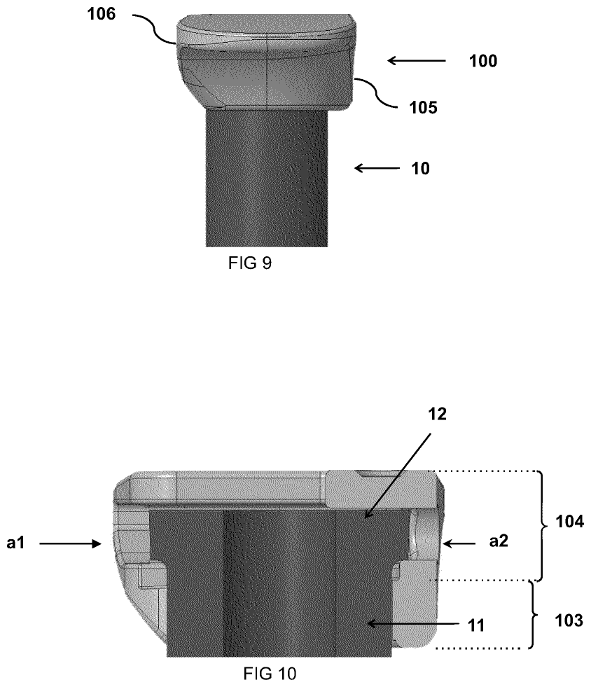

FIG. 5 provides a detailed side view the device focused around the finger grip part 100 as associated to the proximal end 12p of the barrel 12 and flange 12 in a fully engaged position. FIG. 2 provides a cross section bottom view of an injection device just below the collar, whereby the plunger has been omitted so as not to obstruct detailed view. FIG. 4 provides a corresponding top view. FIG. 3 provides a top view of the finger grip in empty form (without syringe inserted). FIG. 6 provides a view of the configuration illustrated in FIG. 5 without syringe. FIGS. 7 and 8 provide top and bottom perspective views of the empty grip. FIGS. 9 and 10 respectively provide a side view and cross section side view of the configuration shown in FIG. 5.

As indicated in FIG. 6 the grip 100 comprises a cavity 150 to receive the syringe. The cavity can be understood as comprising a first portion 151 (berth) for receiving the flange 12 of the syringe and a second cavity portion 152 (berth), that is fluidly connected to first, for receiving the barrel 11 along its terminal end 11p. As indicated, the first portion of the cavity 151 can be understood as being defined between opposing inner surfaces 110, 120 of the base part 104 of the finger grip 100. The second portion of the cavity 152 for receiving the barrel of the syringe is bound laterally by an inner surface 130 of a collar portion 103 that extends downward from wall portions bounding the first cavity part in a direction along central axis “A”. The collar typically extends over a length of at least 2 mm, Typically in a range of 2-10 nm, e.g. 3-6 mm such as about 5 mm. The longer the collar part the more stable the connection with a syringe can be. To minimize impact on gas flow resistance between collar and barrel the overlap is preferably≤10 mm.

It will be appreciated that the grip advantageously provides an open structure to maximize gas access to the syringe. To this end the walls bounding the cavity can be provided with one or more aperture. As best seen in FIGS. 6-10 cavity 151 can be understood as being bound sideways by opposing sidewalls 105,106. These sideways typically do not engage with the flange or any other part of the syringe so as to minimize flush contact area. At least one of the sidewalls 105 includes a first aperture al dimensioned to receive the flange from a sideways direction. The opposing sidewall 106 can be provided with a second aperture a2, typically comparatively smaller than the first, to allow gas passage through the cavity. The wall defining the top of cavity 151 includes a further aperture a4 that is shaped to accommodate passage of the plunger rod. The collar is typically shaped/dimensioned to partially wrap around a perimeter of the barrel leaving an aperture a3 dimensioned to allow inserting the barrel from the side-ways direction.

Similar to the walls bounding the first part of the cavity 151 the collar may include one or more further openings to further improve gas passage across the cavity. Apertures a1 through a4 are indicated in FIGS. 7, 8 and 10. FIG. 3 and FIG. 7 also indicate a residual mark m from injection moulding.

The grip comprises a plurality of contacts C1-1, C1-2, C1-3, C1-4, C2-1,C2-2, C2-3, C2-4, C2-5 for engaging with opposing points along the external surface of the syringe. In each of the embodiments presented herein below the first set of contacts C1 (for engaging with the flange) and the second set of contacts C2 (for engaging with the barrel) are embodied in a form of contact lines. Note that, as depicted. inner faces 110, 120 and 130 are essentially without protrusions.

The contact lines can be provided by a number of ways. In a variation, e.g. as best seen in FIGS. 11A, 11B, 12A, 12B, 13, 14, 15, and 16, the contacts can be formed by one or more of: a tangential contact between an exterior surface of the barrel or the flange of the syringe and one facet (e.g. at a central portion thereof) of a multifaced portion of the interior surface of the base part or the collar part; by a contact between an exterior surface of the barrel or the flange of the syringe and one of an apex of a bend (or fold) of the interior surface of the base part or of the collar part; and/or by a bend or an intersect between two facets of a multifaced portion of the interior surface of the base part or the collar part fold or bend.

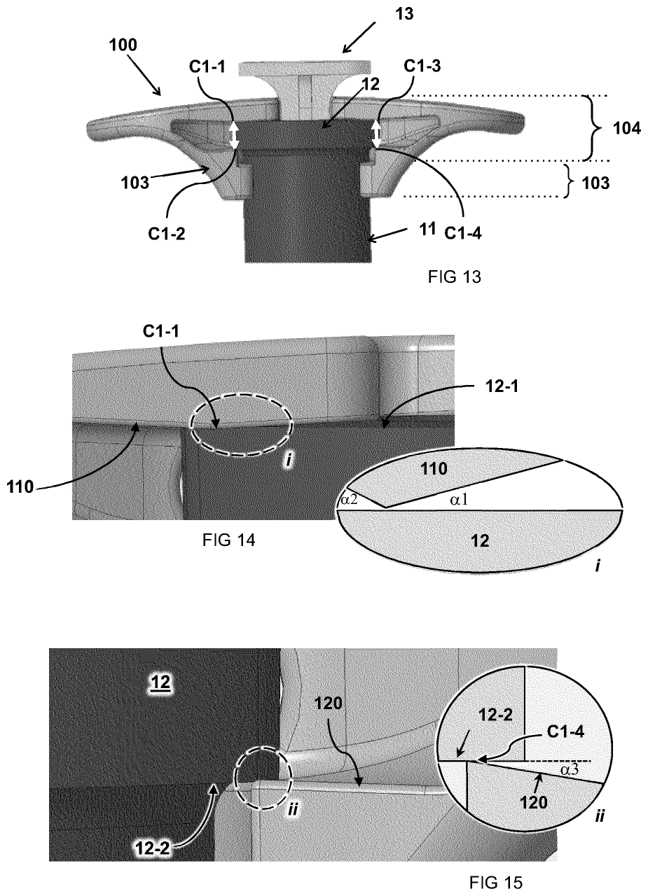

As indicated in FIGS. 13-15 a line contact can be realized by portions of the inner surface that are oriented under an angle a (a1, a2, a3), relative to the upper and lower faces of the flange to be received. The angle is typically in a range between 1 and a maximum of 45°. Preferably, the angle is in a range of about 5 to about 20 degrees. Upon coupling of the syringe to the grip essentially line-shaped contacts are formed. An angle in the specified range was found to provide sufficient gas access. In some embodiments, e.g. as indicated in FIGS. 14-15, the inner face may be comprised of two or more sloped areas that meet at an intersect providing a line shaped apex. Note that the slope in schematics ‘i’ and ‘ii’ may be exaggerated for clarity.

The contact between the inner face 130 of the collar 103 and the barrel 11 of the syringe is realized without protrusions. A protrusion free contact can be realized in a number of ways.

For example, line shaped contacts can be provided by a berth for the barrel (cavity portion 152) having a polygonal-shaped cross section. As opposed to providing protrusions an inner face if the collar 103 can be configured to comprise a plurality of faces that are arranged under an angle relative to each other. Upon inserting the barrel some or each of the planar faces can form a tangential contact with the barrel. The number of planes is ≥3. In one embodiment, e.g. as best seen in FIGS. 11A and 17B, the collar comprises 5 planar faces 130-1,130-2,130-3,130-4,130-5 (see FIG. 17) that are oriented to provide what somewhat resembles a hexagonal-shaped barrel berth which is open along one side. Because the barrel has a rounded shape overall flush contact area can be negligible.

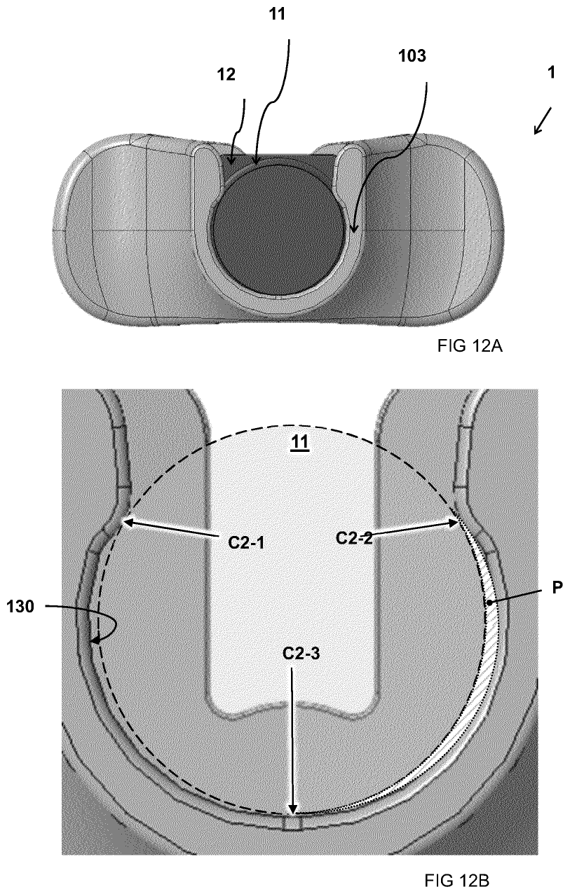

In another or further variant the barrel berth can be oblong shaped. As best seen in FIG. 12B inner face 130 of the collar can be essentially smooth. Upon coupling with a syringe the inner face of the collar 103 forms three tangential contacts c2-1,c2-2,c2-3 with the barrel 11. The total area of contact between barrel and the collar can be negligible. Note the gas passages P between the barrel and the collar.

It will be understood that the barrel berth is not to be understood as being limited to the depicted configurations, other polygons or smooth curved cross sections (e.g. square, octagon, elliptical, etc.) are envisioned.

FIG. 16 details a variation wherein the grip is configured to provide a loose contact with the barrel. Specifically, the collar is somewhat over-dimensioned with respect to a cross-sectional dimension of the barrel to be received. In particular planar contact faces 130-1 to 130-5 are positioned so that a maximum of three simultaneous contacts can be formed between the collar and the barrel. In FIG. 16 the barrel is indicated in an extreme position wherein two simultaneous contact lines c2-1,c2-2 are realized. Along opposing ends there is some space or slack ‘s’.

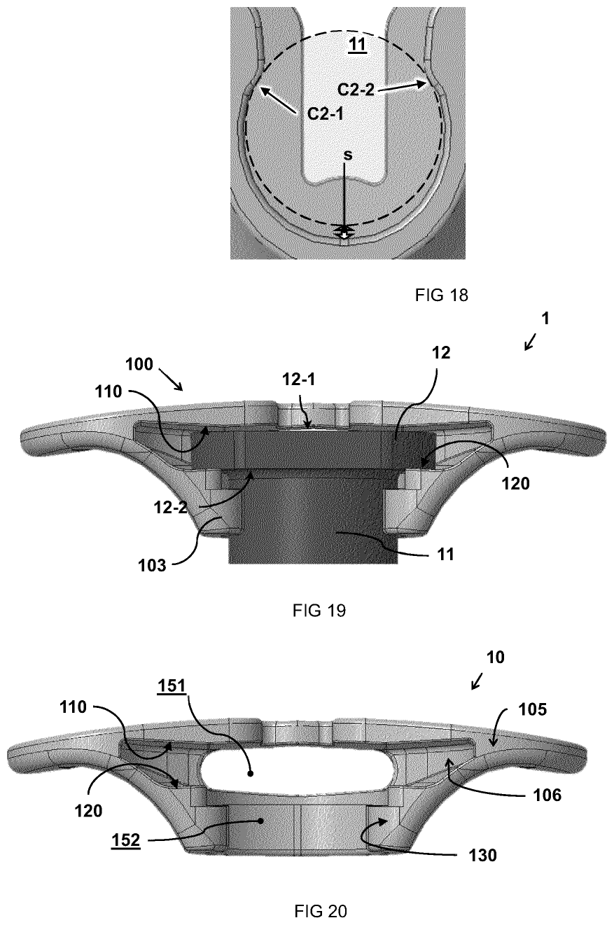

FIG. 18 details a further variation wherein the grip is configured to provide a loose contact with the barrel. As compared to the variation shown in FIG. 12A-B the berth is oblong shaped so that only two simultaneous contacts with the barrel 11 can be realized upon full coupling.

The somewhat loose fit improves gas access between the barrel and finger grip. Typically the slack is ≥1 μm, e.g. ≥5 μm. Generally slack is ≤300 μm, e.g. ≤200 μm, e.g. between 5-300 μm or between 1-200 μm.

Inventors found that within the specified range the slack is essentially unnoticeable by an end-user during normal handling operations.

Of course the grip can be configured wherein the berth matches an external diameter of the barrel to be received (cf. FIGS. 11, 12 and 17) yielding a total of five contact, respectively three line contacts with the barrel 11.

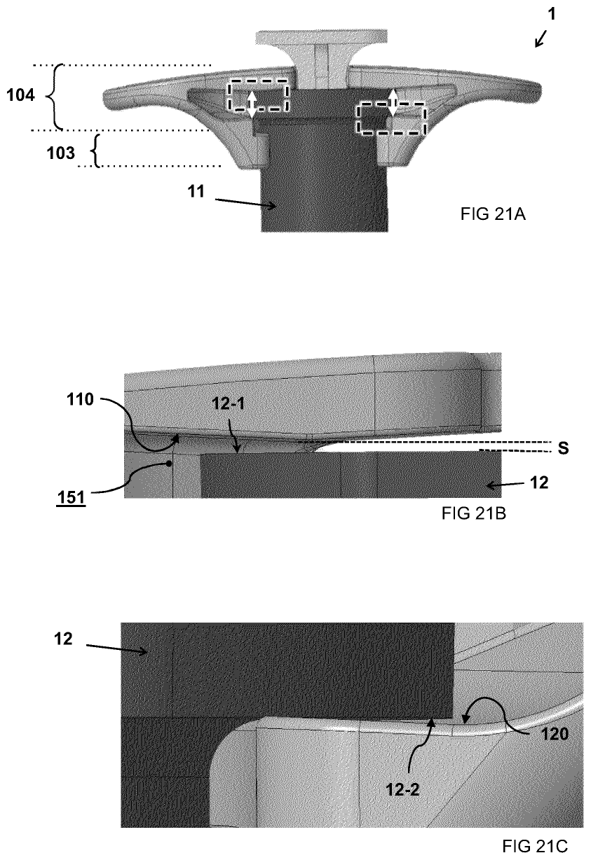

FIGS. 19, 20 and 21 detail aspects of configurations wherein the grip is dimensioned so that a coupling with the flange is looser. FIGS. 19 and 21A to 21C provide partial side views of an injection device 1 for administering a drug. FIG. 20 illustrates the same grip but without syringe.

As seen, the cavity part 151 for receiving the flange as defined between inner faces 110 and 120 is over-dimensioned with respect to a thickness of the flange 12 to be received. In the embodiment shown the syringe is illustrated with the lower face 12-2 of flange 12 engaged with inner face 120. Accordingly, space or slack S is available between the upper face 12-1 and face 110. The slack improves gas access between flange and finger grip. Typically slack is ≥1 μm, e.g. ≥5 μm. Generally slack is ≤400 μm, ≤300 μm, e.g. ≤200 μm, e.g. between 5-300 μm or between 1-200 μm. Inventors found that within the specified range is essentially unnoticeable by an end user during normal handling operations.

The over-dimensioning of the barrel berth and/or the flange berth advantageously accommodates common syringe manufacturing tolerances. The grip can advantageously be manufactured, even as a single piece, using conventional manufacturing methods including but not limited to moulding, e.g. injection moulding. Without wishing to be bound by theory the eased manufacturing is believed to be at least in part related to the provision of smooth inner faces of the grip as disclosed herein. Specifically the absence of outwardly extending protrusions (ribs, piles, domes) is believed to facilitate filling of the mould and release of the formed product from the mould.

The method comprises: forming a plastic compound, typically a thermoplastic compound, using a mould configured to define: a base part of the grip defining a first portion of a cavity of the grip for receiving the flange, a collar part of the grip defining a second potion of the cavity of the grip, for receiving an end portion of the barrel adjacent the flange, and at least a first and second finger rest extending laterally from the grip, wherein the mould is configured such that an inner surface of the base part bounding the first portion of the cavity and an inner surface of the collar bounding the second portion of the cavity are formed such as to respectively provide a first set of first contacts for engaging with an exterior surface of the flange, and a second set of second contacts for engaging with an exterior surface of the barrel, and wherein the mould is configured such that the inner surface of the base part of the grip and the inner surface of the collar part of the grip are smooth or at least free of outwardly extending protrusions for contacting portions of the syringe.

Advantageously, the injection device as disclosed can be prefilled with a medicament, e.g. an ophthalmic medicament.

The medicament can advantageously be packaged, wherein an exterior of the injection device is sterilized, whereby the syringe can be empty or prefilled.

The packaged injection device can be provided by a method comprising: a) providing a syringe comprising a barrel and a flange; b) filling the syringe with a medicament, if so desired; c) providing the finger grip comprising: a base part defining a first portion of a cavity of the grip for receiving the flange, a collar part defining a second potion of the cavity of the grip, for receiving an end portion of the barrel adjacent the flange, and at least a first and second finger rest portions extending laterally from the grip, wherein an inner surface of the base part bounding the first portion of the cavity and an inner surface of the collar bounding the second portion of the cavity are formed such as to respectively provide a first set of first contacts for engaging with an exterior surface of the flange, and a second set of second contacts for engaging with an exterior surface of the barrel, and wherein the inner surface of the base part of the grip and the inner surface of the base part of the grip are smooth or at least free of outwardly extending protrusions for contacting portions of the syringe, ; d) coupling the finger grip to the syringe forming an injection device comprising at least the syringe and the finger grip coupled with the syringe; e) sterilizing an exterior of the injection device by a process comprising an exposure to a gaseous sterilizing agent; f) placing the injection device in a package; and g) sealing the package. Advantageously the, sterilizing can be performed after the filling and/or after the coupling. Optionally, the sterilizing, or a further sterilizing, can comprise an exposure to a gaseous sterilizing agent. Yet further, the sterilizing optionally can be performed with the product already partly or fully placed in the package.

For the purpose of clarity and a concise description, features are described herein as part of the same or separate embodiments, however, it will be appreciated that the scope of the invention may include embodiments having combinations of all or some of the features described. Of course, it is to be appreciated that any one of the above embodiments or processes may be combined with one or more other embodiments or processes to provide even further improvements in finding and matching designs and advantages.

In interpreting the appended claims, it should be understood that the word “comprising” does not exclude the presence of other elements or acts than those listed in a given claim; the word “a” or “an” preceding an element does not exclude the presence of a plurality of such elements; any reference signs in the claims do not limit their scope; several “means” may be represented by the same or different item(s) or implemented structure or function; any of the disclosed devices or portions thereof may be combined together or separated into further portions unless specifically stated otherwise. Where one claim refers to another claim, this may indicate synergetic advantage achieved by the combination of their respective features. But the mere fact that certain measures are recited in mutually different claims does not indicate that a combination of these measures cannot also be used to advantage. The present embodiments may thus include all working combinations of the claims wherein each claim can in principle refer to any preceding claim unless clearly excluded by context.

1. An injection device comprising:
a syringe having a barrel and a flange; and
a finger grip coupled with the syringe,
the finger grip comprising:
i) a base part defining a first portion of a cavity of the grip receiving the flange,
ii) a collar part defining a second portion of the cavity of the grip, receiving an end portion of the barrel adjacent to the flange, and wherein,
an inner surface of the base part bounding the first portion of the cavity and an inner surface of the collar bounding the second portion of the cavity are formed such as to respectively provide a first set of first point and/or line contacts for engaging with an exterior surface of the flange, and a second set of second point and/or line contacts for engaging with an exterior surface of the barrel, wherein
the inner surface of the base part of the grip and the inner surface of the base part of the grip are smooth or at least free of outwardly extending protrusions for contacting portions of the syringe, over zones that least encompass the contacts comprised in the first and the second set.

2. The device according to claim 1, wherein engagement between the grip and the syringe is provided exclusively by the contacts or a subset thereof as comprised in first and second set of contacts.

3. The device according to claim 1, wherein the first and the second contacts are comprised of line contacts.

4. The device according to claim 1, wherein there is a tight coupling between the flange of syringe and the grip by the base part being configured to provide a simultaneous direct contact of each the first contacts comprised in the first subset with the exterior surface of the flange, and wherein there is a looser coupling between the barrel of syringe and the grip by the collar part being configured such that only limited subsets of the second contacts comprised in the second set can be in a simultaneous direct contact with the barrel during normal operation.

5. The device according to claim 1, wherein there is a tight coupling between the barrel of syringe and the grip by the collar part being configured to provide a simultaneous direct contact of each the second contacts comprised in the second subset with the exterior surface of the barrel, and wherein there is a looser coupling between the flange of syringe and the grip by the base part being configured such that only limited subsets of the first contacts comprised in the first set can be in a simultaneous direct contact with the flange during normal operation.

6. The device according to claim 4, wherein a maximum slack between non-engaging first contacts and the flange or non-engaging second contacts and the barrel is in a range of 50-400 μm.

7. The device according to claim 1, wherein the contact lines are provided by one or more of:
a tangential contact between an exterior surface of the barrel or the flange of the syringe and one facet of a multifaced portion of the interior surface of the base part or the collar part, or
a contact between an exterior surface of the barrel or the flange of the syringe and one of an apex of a bend of the interior surface of the base part or the collar part fold or bend or an intersect between two facets of a multifaced portion of the interior surface of the base part or the collar part fold or bend.

8. The device according to claim 1, wherein the number of first contacts for engaging the flange equals four.

9. The device according to claim 1, wherein the number of second contacts for engaging the barrel is between 3 and 5.

10. The device according to claim 9, wherein the number of second contacts for engaging the barrel is 3.

11. The device according to claim 1, wherein the grip further comprises a backstop tab to restrict release of a plunger from the barrel.

12. The device according to claim 1, wherein the grip is an integrally formed part.

13. The device according to claim 1, wherein the syringe is filled with a medicament.

14. A packaged injection device comprising the injection device according to claim 1, wherein an exterior of the injection device is sterilized.

15. A finger grip configured for coupling with a syringe comprising a barrel and a flange, said finger grip comprising:
a base part of the grip defining a first portion of a cavity of the grip for receiving the flange, and
a collar part of the grip defining a second potion of the cavity of the grip, for receiving an end portion of the barrel adjacent the flange,
wherein an inner surface of the base part bounding the first portion of the cavity and an inner surface of the collar bounding the second portion of the cavity are formed such as to respectively provide a first set of first point and/or line contacts for engaging with an exterior surface of the flange, and a second set of second point and/or line contacts for engaging with an exterior surface of the barrel, and
wherein the inner surface of the base part of the grip and the inner surface of the base part of the grip are smooth or at least free of outwardly extending protrusions for contacting portions of the syringe, over zones that least encompass the contacts comprised in the first and the second set of contacts.

16. A method of manufacturing a finger grip configured for coupling with a syringe comprising a barrel and a flange; said method comprising forming a plastic compound using a mould configured to define:
a base part of the grip defining a first portion of a cavity of the grip for receiving the flange, and
a collar part of the grip defining a second portion of the cavity of the grip, for receiving an end portion of the barrel adjacent the flange,
wherein the mould is configured such that an inner surface of the base part bounding the first portion of the cavity and an inner surface of the collar bounding the second portion of the cavity are formed such as to respectively provide a first set of first point and/or line contacts for engaging with an exterior surface of the flange, and a second set of second point and/or line contacts for engaging with an exterior surface of the barrel, and
wherein the mould is configured such that the inner surface of the base part of the grip and the inner surface of the base part of the grip are smooth or at least free of outwardly extending protrusions for contacting portions of the syringe, over zones that least encompass the contacts comprised in the first and the second set of contacts.

17. A method of manufacturing a packaged injection device, said method comprising:
a) providing a syringe comprising a barrel and a flange;
b) filling the syringe with a medicament;
c) providing the finger grip comprising: a base part defining a first portion of a cavity of the grip for receiving the flange, and a collar part defining a second potion of the cavity of the grip, for receiving an end portion of the barrel adjacent the flange, wherein an inner surface of the base part bounding the first portion of the cavity and an inner surface of the collar bounding the second portion of the cavity are formed such as to respectively provide a first set of first point and/or line contacts for engaging with an exterior surface of the flange, and a second set of second point and/or line contacts for engaging with an exterior surface of the barrel, and wherein the inner surface of the base part of the grip and the inner surface of the base part of the grip are smooth or at least free of outwardly extending protrusions for contacting portions of the syringe, over zones that least encompass the contacts comprised in the first and the second set of contacts;
d) coupling the finger grip to the syringe forming an injection device comprising at least the syringe and the finger grip coupled with the syringe;
e) sterilizing an exterior of the injection device by a process comprising an exposure to a gaseous sterilizing agent;
f) placing the injection device in a package; and
g) sealing the package,
wherein the sterilizing is performed after the filling and the coupling.

18. The method according to claim 17, wherein the sterilizing e) or a further sterilizing comprising an exposure to a gaseous sterilizing agent is performed after e).

19. The device according to claim 5, wherein a maximum slack between non-engaging first contacts and the flange or non-engaging second contacts and the barrel is in a range of 50-400 μm.

20. The device according to claim 6, wherein a maximum slack between non-engaging first contacts and the flange or non-engaging second contacts and the barrel is in a range of 50-300 μm.
